(19) United States
(12) Patent Application Publication
(10) Pub. No.: US 2005/0260542 A1
(43) Pub. Date: Nov. 24, 2005

(54) Arrangement for implants bearing growth-stimulating substance or substances, and one such implant

(75) Inventor: Jan Hall, Goteborg (SE)

Correspondence Address: CONNOLLY BOVE LODGE & HUTZ LLP, SUITE 800, 1990 M STREET NW, WASHINGTON, DC, 20036-3425, US

(73) Assignee: Nobel Biocare AB (publ.), Goteborg (SE)

(21) Appl. No.: 10/520,759

(22) PCT Filed: Jun. 26, 2003
(86) PCT No.: PCT/SE03/01108

§ 371 Date: Jan. 11, 2005

(51) Int. Cl. A61C 8/00; A61B 17/86

(52) U.S. Cl. 433/174; 606/73; 424/425

(57) ABSTRACT

An implant bears growth-stimulating substance(s), here called GSS, which can be released when the implant is arranged in a jaw bone hole. The release takes place in interaction with secreted, cell-containing body fluid so as to form new bone alongside the implant. The implant is arranged with an outer surface which can comprise the outer parts of a thread or can consist of bearing surfaces parallel to the inner wall of the jaw bone hole. First portions of the outer surface have first diameters or radii, and second portions have second diameters or radii smaller than the first diameters or radii. The implant bears against or cooperates with the hole wall via the first portions and, by means of the second portions and together with the hole wall, forms one or more closed spaces into which body fluid can penetrate and GSS can be released. The implant can alternatively be designed with a shape or shapes substantially corresponding to the tooth root configuration(s). The new bone formation is made effective in this way, and great stability can be obtained for the implant in its position in the jaw bone.

The present invention relates to an arrangement for implants bearing growth-stimulating substance or substances, here called GSS, which, when the implant is in its position fitted in a jaw bone, is arranged to interact with cell-containing body fluid secreted at the jaw bone and thus form new bone alongside the implant.

The invention also relates to an implant which can be fitted in a jaw bone hole created by hole formation or tooth root extraction.

In connection with implants fitted in jaw bone holes, it is already known to use GSS in combination with calcium phosphates, autologous bone, allogenic bone, etc., for bone formation purposes. The bone formation is used to increase the stability of the implant in the jaw bone. Depending on the jaw bone status and the implant position, it is possible to use different types, amounts and concentrations of substances and materials of said type. It is known, inter alia, to use GSS, for example, in conjunction with implants of the self-tapping type or implants with a thread intended to be screwed into a thread formed in the jaw bone.

In this connection, reference may be made to patent applications SE 9901972-1 and SE 9901973-9 submitted by the same Applicant and with the same inventor as in the present application.

Reference may also be made to the article published by, inter alia, the inventor of the present patent application and entitled “Properties of a New Porous Oxide Surface on Titanium Implants, Volume 1: The Oxidized Titanium Surface, Applied Osseointegration Research”.

The present invention is based on the recognition that new bone formation is at its most effective in arrangements where space can be created for secreted body fluid and released GSS. The object of the present invention is to solve this problem among others, and the invention proposes, inter alia, that threaded implants known per se will be able to be used for creating an effective process of new bone formation alongside the implant.

There is also a need to make available methods and arrangements which permit extended possibilities of implant positioning, giving improved results from the point of view of appearance and/or stability. The invention also solves this problem.

There is also a need for implants of new designs which can be fitted by a new method, for example a method in which they are pressed in manually. These new methods and implants are especially desirable when a hole has been formed by tooth root extraction, possibly in combination with subsequent finishing of the jaw hole.

The feature which can principally be regarded as characterizing an arrangement according to the invention is, inter alia, that the implant is arranged with an outer surface which can be placed against a wall of the jaw bone hole and which is arranged with first portions of first diameters or radii and second portions of second diameters or radii smaller than the first diameters and radii, and that the implant bears against or cooperates with the hole wall via the first portions and, by means of the second portions and together with the hole wall, forms one or more closed spaces into which body fluid can penetrate and GSS can be released for interaction with cells, for example stem cells, in the body fluid.

In further developments of the invention, it is proposed that the outer surface that can be placed against the wall of the jaw bone hole will be provided with one or more threads or thread sections and that the first portions comprise the thread crests or thread outer parts of the threads or thread sections. The second portions can in this case comprise the bottom parts or inner parts of the threads, and said closed spaces are thus situated between the thread crests or thread outer parts. Each thread can extend along all or most of the circumference of the implant. The outer surface or the thread or threads can be provided with porous layers, by means of which GSS can be stored on the implant. The implant can thus bear most (the greater concentration) of the GSS at said closed spaces and the implant can be grafted with GSS, which can give a skin with a thickness of a few nanometers. In general, the thickness can be chosen at from a few Ångström to a few micrometers. The first portions can be arranged to cooperate with the hole wall which affords initial positional stability for the implant in the jaw bone. Thus, the first portions can have a degree of penetration into the jaw bone in the range of 5-20% of the height of the first portions above the second portions. The outer threads or outer parts can thus be arranged with a thread depth which on the one hand affords a screwing-in function in the hole wall in the jaw bone and on the other hand provides for formation of said closed space. In an alternative embodiment, the implant is designed with longitudinal and/or transverse recesses which are charged or provided with GSS in possible cooperation with material or substitute of the aforementioned type.

The feature which can principally be regarded as characterizing an implant according to the invention is, inter alia, that it is on the one hand provided with osteoinductive material in the form of growth-stimulating substance(s) or GSS arranged to interact with cells (for example stem cells) present in the body fluid so that new bone is formed, and, on the other hand, its inner parts are configured in close or substantial approximation to the line(s) of the hole in the jaw bone at its inner parts.

In a preferred embodiment, the implant is designed to extend or branch substantially like the tooth root of the tooth. At its inner parts, the implant can be curved in relation to the main longitudinal extent of the implant. The implant can be designed with two or three parts arranged, for example curved, in relation to the main direction. One or more spaces can be present between the inner parts of the implant and the wall(s) of the hole, in which space or spaces said interaction is intended to take place. At its inner branching parts, the implant bears growth-stimulating substance(s) on the outer surfaces of the branches. The implant can be fitted in the jaw bone hole by means of a downwardly or inwardly directed pressing force applied to the implant, preferably of a manual nature, and the outer surface(s) of each branch at the implant's inner parts can be provided with a surface roughness or porous oxide layer, by means of which GSS can be applied in layers of identical or varying concentration.

By means of what has been proposed above, an implant is obtained which, through the use of GSS, possibly in combination with substitute or material of said type, gives an improved new bone growth, at the same time with possible use of implants known per se and techniques for application of GSS, which for example can be stored on the implant in porous outer oxide layers. By means of the proposed arrangement, relatively large amounts and concentrations of GSS can be applied to the implant, for example to its outer surface. A new technique of fitting the implant in the jaw bone hole is made possible. The implant is quite simply pressed manually into the hole and the GSS used in the arrangement of spaces allows the implant to be maintained and anchored in the jaw bone. GSS can also be applied to the outer surface in the form of a gel to which GSS has been added. GSS may if appropriate be mixed with calcium phosphate(s), autologous bone, allogenic bone, xenografts, etc.

A presently proposed embodiment of an arrangement having the features characteristic of the invention will be described below with reference to the attached drawing, in which

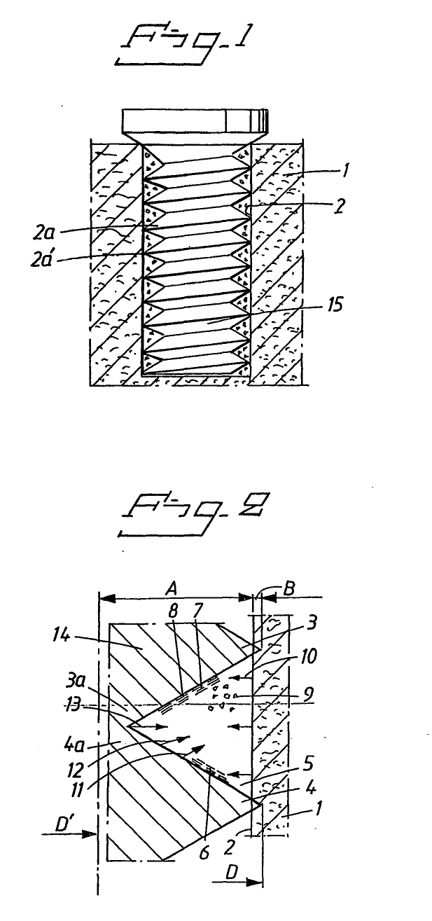

FIG. 1 shows, in vertical section, an implant fitted in a jaw bone hole and provided with an outer thread,

FIG. 2 shows, in a vertical view and enlarged in relation to FIG. 1, the cooperation of the outer thread with the wall of the jaw bone hole, and grafting-on of GSS in combination with material or substitute of other types,

FIG. 3 shows, in a vertical view and again enlarged, an application, differing from FIG. 2, of a skin of GSS in a space created by inner parts of an implant thread and the hole wall in the jaw bone,

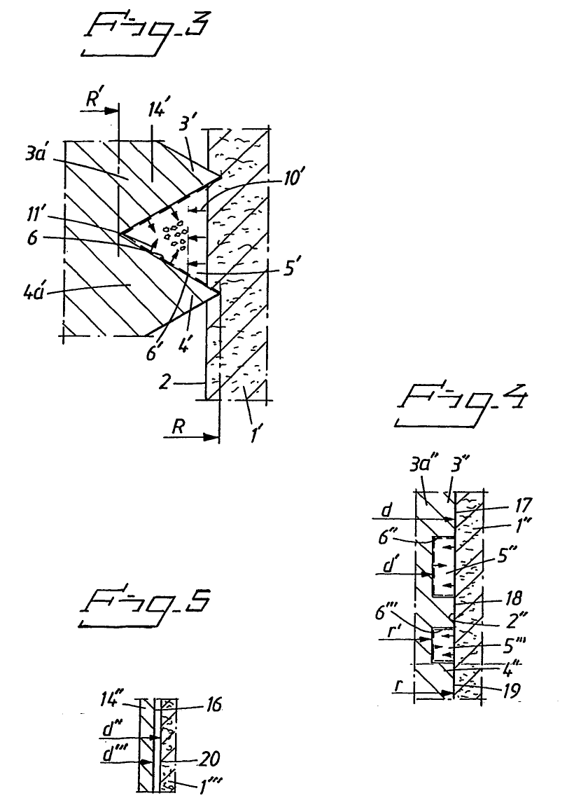

FIG. 4 shows, in a vertical view, a configuration of the implant's outer surface different than the thread form, and the way in which this configuration bears on and cooperates with the wall in the jaw bone hole,

FIG. 5 shows, in a vertical view, an alternative embodiment with recesses extending along the height of the implant, and

FIGS. 6-9 show tooth and implant configurations in vertical views, and, in block diagram form, the production of an implant.

In FIG. 1, a jaw bone is symbolized by 1. The jaw bone is provided with a jaw bone hole 2 created in a manner known per se with a drill. The implant can be designed in a manner known per se with an outer thread 2a. The implant can also be treated so that a porous oxide layer is present on the outsides of the threads. Said oxide layer can provide a storage function for GSS, and the latter can consist of matrix molecules, growth factors and differentiation factors and/or peptides with growth-stimulating properties. The thread crests 2a′ can be placed against the inner wall of the jaw bone hole 2.

FIG. 2 shows, on an enlarged scale in relation to FIG. 1, two thread crests 3 and 4 of the implant. The implant can thus be regarded as comprising first portions with a diameter D and second portions with a second diameter D′, the diameter D exceeding the diameter D′ by a value which corresponds to two thread depths A. In the illustrative embodiment shown, the thread crests at 3, 4 penetrate slightly into the jaw bone 1, the depth of penetration being indicated by B. The depth of penetration can in this case be of an order which means that the implant is afforded a certain initial stability in its position fitted in the hole 2. The depth of penetration B can be in the range of 5-20% of the thread depth A. In the arrangement shown in FIG. 2, a space 5 is thus obtained between each thread pair. The implant is provided with GSS which can be arranged with different concentrations, symbolized by broken lines 6, 7 and 8 on one another. Body fluid 9 containing cells, for example containing stem cells, penetrates into the space 5, and said GSS is released, together with any other substitute or material, so as to effect an interaction and thus form new bone in the space 5. The direction of penetration of the body fluid is symbolized by 10 in FIG. 2, and the directions of release of GSS and possible substitutes or materials are symbolized by arrows 11, 12, 13. The concentrations or amounts 6, 7, 8 can in this case be arranged so that they decrease toward the crests 3, 4 and increase rearward into the space.

FIG. 3 shows an embodiment with only GSS, and where the application of GSS is distributed along the extent of the thread parts. In FIG. 3, the components corresponding to FIG. 2 are indicated with the same reference numbers, with addition of prime markers. In the present case, the radii R and R′ have been indicated in FIG. 3 instead of the diameters D and D′.

FIG. 4 shows an alternative embodiment in which the threads have been replaced by portions 3″, 3′″ which, with circular straight surfaces, bear against the wall of the hole 2″. In this case, recesses 5″ and 5′″ have been formed extending in the circumferential direction. The design of the recesses can be varied and, for example can have internal sphere or arc shapes. Examples of rectangular and square vertical sections for the recesses 5″ and 5′″ have been shown in the figure. In the recesses, the skin/layer/concentration or amount of GSS are indicated by 6″ and 6′″. In this case too, GSS may possibly be combined with substitute or material, in accordance with the above. The portions of greater diameter d or greater radius r are shown together with second portions d′ and r′, respectively.

In FIGS. 2, 3 and 4, the implant has the reference numbers 14, 14′ and 14″. In FIG. 1, the implant has the reference number 15. In FIG. 5, the implant 14″ is provided with grooves 16 which extend in the height direction and which can be arranged in parallel along the whole circumference of the implant 14′. In an alternative embodiment, the grooves 16 are helical or configured with a zigzag pattern for example. The diameters of the first portions are indicated by d″, and the diameters of the second portions are indicated by d′″. In FIGS. 2, 3 and 4, rear portions have been indicated by 3a, 4a; 3a′, 4a′; and 3a″, 4a″. Each thread can extend about the whole or most of the periphery or circumference of the implant. In FIGS. 4 and 5, reference numbers 17, 18, 19 and 20 indicate surface portions parallel to the hole wall placed against the implants by clamp fitting. At least at the portions 17, 18, 19, 20, the implant is designed with a surface roughness or porous layers which can bear GSS of identical or different concentrations and amounts.

In FIG. 6, a jaw bone is indicated diagrammatically by 21. A tooth in the jaw bone is indicated by 22 and the tooth is in this case of the type which has two root parts 22a and 22b. The tooth extends in the jaw bone hole 23 which is shown with an overdimensioned gap for reasons of clarity. The tooth can be extracted from the jaw bone in the direction of arrow 24.

FIG. 6a shows alternative configurations of the root parts 22a′ and 22b′.

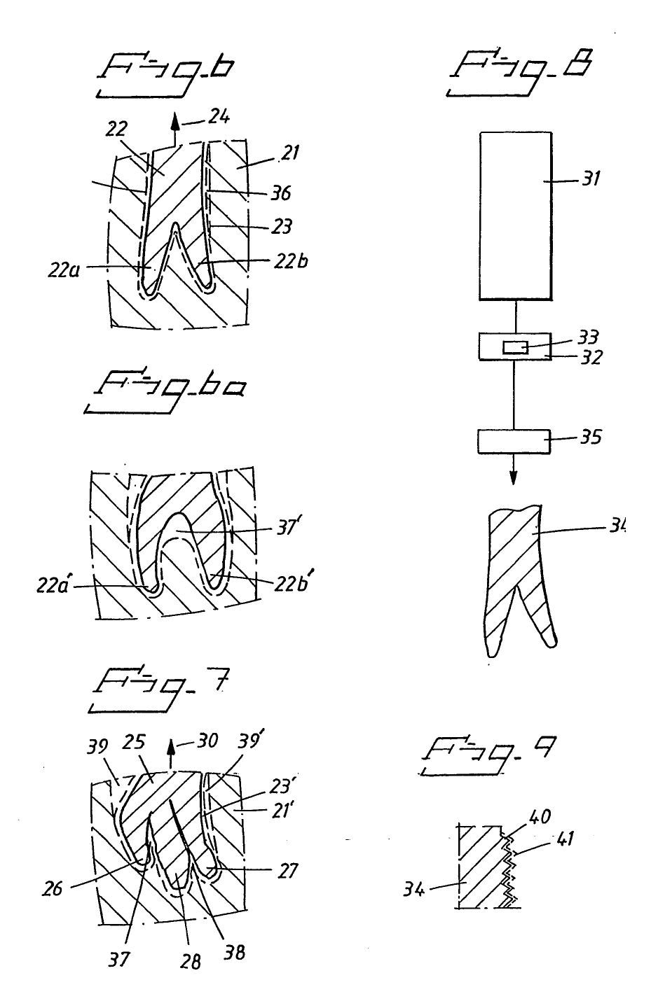

FIG. 7 shows the lower parts of a tooth 25 provided with three root parts 26, 27 and 28. The jaw bone is in this case indicated by 21′. The tooth 25 in question can be extracted from the jaw bone in the direction of arrow 30. The hole 23 in the jaw bone for this tooth is shown with an overdimensioned gap for reasons of clarity.

Upon extraction of the tooth 22 according to FIG. 6 together with the root and all, the jaw bone hole 23 acquires a shape corresponding to that of the tooth. In accordance with FIG. 8, an appliance 31 is used to define or image the jaw bone hole 23 in FIG. 6 when the tooth 22 has been extracted. An imaging technique known per se can be used, for example X-ray, computed tomography, etc. With the appliance 31, the surgeon, the dentist or other person performing treatment is given an image of the shape of the jaw bone hole 23. The shape is assigned a representation in an appliance 32 which can be part of a computer installation known per se. The representation is symbolized by 33 and can be used as a basis for production of an implant 34 which is intended to be placed in the jaw bone hole 23 in question (see FIG. 6). The fitting operation can be carried out in such a way that the implant can be applied with relatively little clearance in the jaw bone hole. The implant 34 can have a design which, upon application of the implant in the jaw bone hole, means that the hole wall springs aside and then back to a position corresponding to the position of the tooth 22 in FIG. 6. Alternatively, the implant can be made to some extent resilient in those parts which upon application are intended to match narrowing parts in the jaw bone hole. The inner parts of the tooth root can also extend in such a way that they together have cross-sectional areas which are smaller than the cross-sectional area or cross-sectional areas of above parts of the jaw bone hole. The implant 34 in question can be produced using production equipment 35 of the PROCERA type. The implant can be milled, cast, or produced in some other way. The implant can be made of titanium, ceramic, etc. The implant can be given an optimum geometric configuration so that the load on the implant is correctly distributed.

It also lies within the possibilities of the invention that the line of the jaw bone hole can be acted upon using tools, for example drilling tools, so that wider parts situated at the bottom can easily match passages in the hole which have been narrower from the start. In FIG. 6, such working is indicated by 36. The space initiated in this way by the recessing or working 36 around the fitted implant can be used as a closed space for new bone formation in accordance with what has been described above. Such working of jaw bone holes can be carried out in different ways from case to case.

In FIG. 7, the spaces 37 and 38 have been formed at the lower parts of the tooth root. In accordance with the above, these spaces 37 and 38 can be used as closed spaces for new bone formation. It will be appreciated that in cases where there is no resiliency function in the jaw bone or implant, said working can allow the tooth roots belonging to the tooth in question to be simulated to a very high degree when producing the implant in question, i.e. the implant 34 in FIG. 8. The root formation according to FIG. 7 can also be completed in a relatively simple manner with working(s) 39, 39′ permitting application of an implant with a configuration which corresponds to the design of the tooth root arrangement according to FIG. 7. The spaces 39, 39′ are also used as closed spaces for new bone formation.

In FIG. 6a, a closed space 37′ for new bone formation is arranged, in accordance with the above, between the original sites of the tooth roots.

FIG. 9 shows that each implant, for example the implant 34, can be provided with surface roughness(es) or porous outer layer(s) 40 for storing GSS 41 of the same amount and/or different concentrations. The surface roughnesses/porosities are arranged at least at the implant's branches 22a, 22b and 26, 27, 28.

Reference may be made here to patent applications submitted to the Swedish patent office on the same day as the present patent application and by the same Applicant and inventor. Said applications have the following titles:
a) “Arrangement for using osteoinductive or bioactive material to induce bone and/or increase the stability of implants in the jaw bone, and an implant intended for this purpose”.
b) “Arrangement for using bioactive or osteoinductive material to build up a bone-based lateral support for implants in the jaw bone”.
c) “Arrangement of two or more implants provided with growth-stimulating substance(s)”.
d) “Arrangement for increasing the stress resistance of implants, and one such implant”

1. An arrangement for implants (15) bearing growth-stimulating substance or substances, here called GSS, which, when the implant is in its position fitted in a jaw bone hole (2), is arranged to interact with cell-containing body fluid (9), for example fluid containing stem cells, secreted at the jaw bone and thus form new bone alongside the implant, characterized in that the implant is arranged with an outer surface which can be placed against a wall of the jaw bone hole and which is arranged with first portions (3, 4) of first diameters (D) or radii (R) and second portions (3a, 4a) of second diameters (D′) or radii (R′) smaller than the first diameters and radii, and in that the implant bears against or cooperates with the hole wall via the first portions and, by means of the second portions and together with the hole wall, forms one or more closed spaces (15, 15) into which body fluid can penetrate and GSS can be released.

2. The arrangement as claimed in patent claim 1, characterized in that the outer surface that can be placed against the wall of the jaw bone hole (2) is provided with one or more threads (3, 4) or thread sections and in that the first portions comprise the thread crests or thread outer parts of the threads or thread sections, the second portions comprise the bottom parts or inner parts of the threads, and said closed spaces (5) are situated between the thread crests or thread outer parts.

3. The arrangement as claimed in patent claim 2, characterized in that each thread extends along all or most of the circumference of the implant.

4. The arrangement as claimed in patent claim 1, characterized in that the outer surface or the thread or threads is/are provided with porous oxide layers, by means of which GSS can be stored on the implant.

5. The arrangement as claimed in patent claim 1, characterized in that the implant bears most of the total quantity of the GSS at said closed spaces (5).

6. The arrangement as claimed in patent claim 1, characterized in that, when the implant is in its position fitted in the jaw bone hole (2), the first portions are arranged with a degree of cooperation with the hole wall which affords an initial positional stability for the implant in the jaw bone.

7. The arrangement as claimed in patent claim 6, characterized in that the first portions (3, 4) have a degree of penetration into the jaw bone in the range of 5-20% of the height of the first portions above the second portions.

8. The arrangement as claimed in claim 1, characterized in that the outer thread(s) or outer parts have a thread depth which on the one hand affords a screwing-in function in the hole wall in the jaw bone and on the other hand provides for formation of said closed spaces (5).

9. The arrangement as claimed in claim 1, characterized in that the implant has longitudinal and/or transverse recesses (16 and 5″, 5′″) which are charged or provided with GSS of identical or different concentrations in possible cooperation with calcium phosphate(s), autologous bone, allogenic bone, xenografts, polymer material, etc.

10. An implant which can be fitted in a jaw bone hole (4) created by hole formation or tooth root extraction, characterized in that it is on the one hand provided with osteoinductive material in the form of growth-stimulating substance(s), here called GSS, arranged to interact with cells present in the body fluid in enclosed spaces at the jaw bone hole so that new bone is formed, and, on the other hand, its inner parts are configured in close or substantial approximation to the line(s) of the hole in the jaw bone at its inner parts.

11. The implant as claimed in patent claim 10, characterized in that the implant is designed to extend or branch substantially like the tooth root of the tooth.

12. The implant as claimed in patent claim 10, characterized in that, at its inner parts, the implant is curved in relation to the main longitudinal extent of the implant.

13. The implant as claimed in patent claim 12, characterized in that the implant is designed with two or three parts arranged in relation to the main direction.

14. The implant as claimed in claim 10, characterized in that, when it is in its position fitted in the hole, one or more spaces can be formed between the inner parts and the wall(s) of the hole, in which space or spaces said interaction is intended to take place.

15. The implant as claimed in claim 10, characterized in that, at least at its inner branching parts, the implant bears growth-stimulating substance(s) on the outer surfaces of the branches.

16. The implant as claimed in claim 10, characterized in that it can be fitted in the jaw bone hole by means of a downwardly or inwardly directed pressing force applied to the implant, preferably of a manual nature.

17. The implant as claimed in claim 10, characterized in that the outer surface(s) of each branch at the implant's inner parts is/are provided with a surface roughness or porous oxide layer, by means of which GSS can be stored in layers of identical or varying concentration.
